(19) United States
(12) Patent Application Publication
(10) Pub. No.: US 2017/0290994 A1
(43) Pub. Date: Oct. 12, 2017

(54) Medical drip bag heater

(71) Applicant: Robert F. Keller, JR., Metamora, IL (US)

(72) Inventor: Robert F. Keller, JR., Metamora, IL (US)

(21) Appl. No.: 14/998,679

(22) Filed: Apr. 11, 2016

(51) Int. Cl. A61M 5/44

(52) U.S. Cl. CPC A61M 5/445; A61M 2205/36; A61M 2205/3368

(57) ABSTRACT

A medical drip bag heater includes a flexible sheet of material configured to wrap around a drip bag, heating wires, velcro strips, and a thermostat control. The sheet of material includes a body portion with two side edges and a bottom flap with a bottom edge. The heating wires are embedded into the sheet of material and configured to electrically connect to a power source through a thermostat control. The velcro strips are fixedly attached to the two side edges and the bottom edge. The thermostat control controls the temperature of the heater and displays the temperature.

### PRIORITY

This application claims priority to and incorporates by reference in its' entirety, U.S. Provisional Patent Application No. 62/386,607, entitled “Medical Drip Bag Heater”, and filed Dec. 7, 2015.

### BRIEF DESCRIPTION OF THE DRAWINGS

FIG. 1 is a schematic view of a medical drip bag heater with a drip bag in an open mode, according to an exemplary embodiment of the invention.

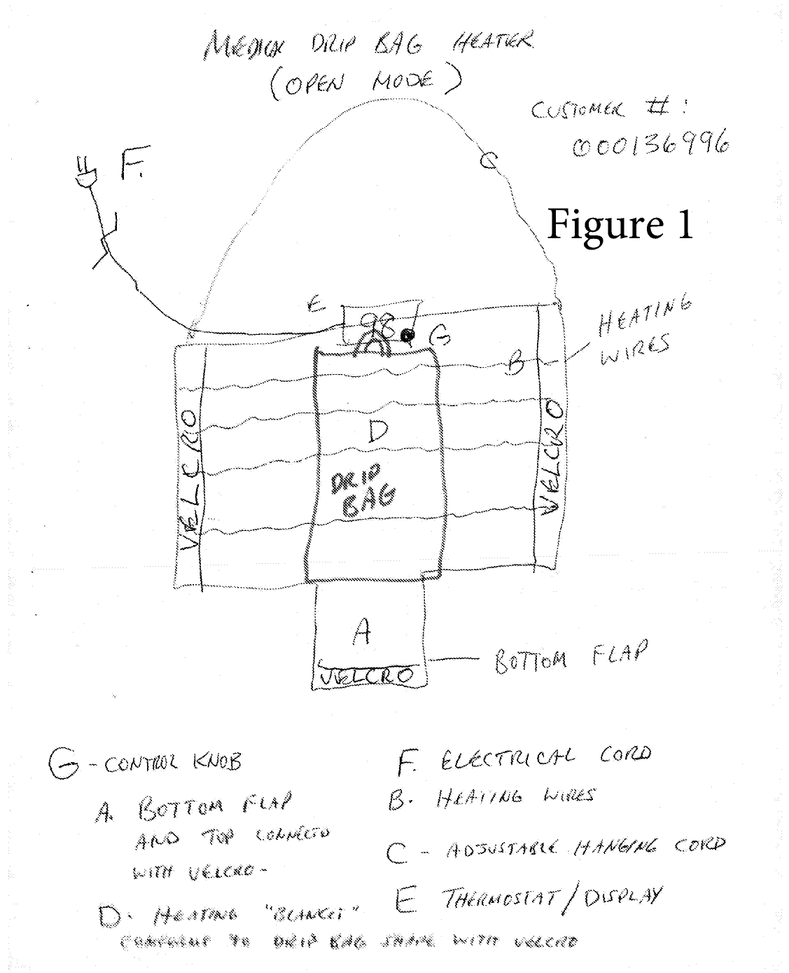

FIG. 2 is a schematic view of a medical drip bag heater with a drip bag in a closed mode, according to an exemplary embodiment of the invention.

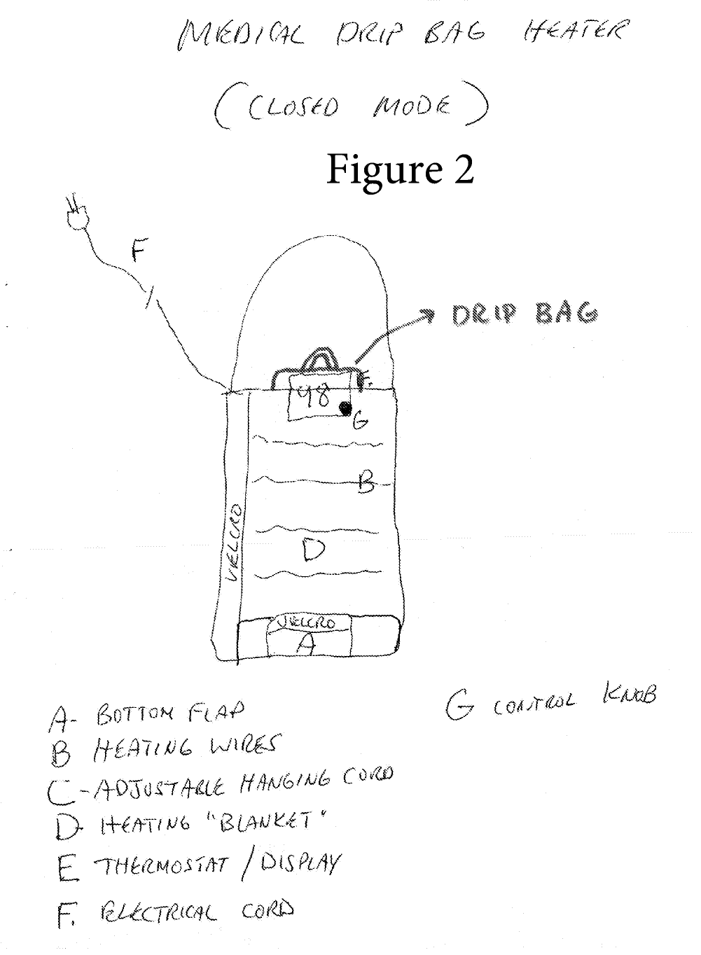

### SPECIFICATIONS: MEDICAL DRIP BAG HEATER

When a drip bag is used to infuse a patient with liquid (e.g., saline solution) it is done at room temperature. Particularly when this is done rapidly (e.g. in the case of dehydration) the patient will usually experience chills from the cooler fluid being rapidly infused. The medical drip bag heater is a small bag similar to an electric blanket which attaches with Velcro and has a small digital display window to display the current temperature. The heater is thermostatically controlled.

I claim:

1. A medical drip bag heater, comprising:
a flexible sheet of material configured to wrap around a drip bag and including a body portion with two side edges and a bottom flap with a bottom edge;
heating wires embedded into the sheet of material and configured to electrically connect to a power source through a thermostat control;
velcro strips fixedly attached to the two side edges and the bottom edge; and
the thermostat control to control a temperature of the heater and display the temperature.
